(19) United States
(12) Patent Application Publication
(10) Pub. No.: US 2013/0281377 A1
(43) Pub. Date: Oct. 24, 2013

(54) TREATING MULTIPLE MYELOMA

(71) Applicants: Rafael Fonseca, Scottsdale, AZ (US); Alexander Keith Stewart, Scottsdale, AZ (US); Peter Bergsagel, Scottsdale, AZ (US); Marta Chesi, Scottsdale, AZ (US)

(72) Inventors: Rafael Fonseca, Scottsdale, AZ (US); Alexander Keith Stewart, Scottsdale, AZ (US); Peter Bergsagel, Scottsdale, AZ (US); Marta Chesi, Scottsdale, AZ (US)

(73) Assignee: MAYO FOUNDATION FOR MEDICAL EDUCATION AND RESEARCH, Rochester, MN (US)

(21) Appl. No.: 13/995,623

(22) PCT Filed: Dec. 22, 2011
(86) PCT No.: PCT/US11/66772

§ 371 Date: Jun. 19, 2013

Related U.S. Application Data
(60) Provisional application No. 61/426,698, filed on Dec. 23, 2010.

(51) Int. Cl. A61K 47/48

(52) U.S. Cl. CPC A61K 47/48284
USPC 514/15.2

(57) ABSTRACT

This document relates to methods and materials for treating multiple myeloma. For example, methods and materials for using albumin-bound paclitaxel composition (e.g., Abraxane®, a paclitaxel albumin-stabilized nanoparticle formulation) to treat multiple myeloma are provided.

### CROSS-REFERENCE TO RELATED APPLICATIONS

This application claims the benefit of U.S. Provisional Application Ser. No. 61/426,698, filed Dec. 23, 2010. The disclosure of the prior application is considered part of (and is incorporated by reference in) the disclosure of this application.

### BACKGROUND

1. Technical Field

This document relates to methods and materials for treating multiple myeloma. For example, this document provides methods and materials for using albumin-bound paclitaxel composition (e.g., Abraxane®, a paclitaxel albumin-stabilized nanoparticle formulation) to treat multiple myeloma.

2. Background Information

Despite aggressive treatment approaches, multiple myeloma can be a fatal B cell malignancy. Multiple myeloma can be recognized clinically by the proliferation of malignant plasma cells in the bone marrow, the detection of a monoclonal protein (M protein) in the serum or urine, anemia, hypercalcemia, renal insufficiency, and lytic bone lesions.

### SUMMARY

This document relates to methods and materials for treating multiple myeloma. For example, this document provides methods and materials for using albumin-bound paclitaxel composition (e.g., Abraxane®, a paclitaxel albumin-stabilized nanoparticle formulation) to treat multiple myeloma. As described herein, administration of Abraxane® to a human patient with multiple myeloma can result in a reduction in the level of free lambda light chain polypeptides in the patient's blood, a reduction in the level of monoclonal IgA polypeptides in the patient's blood, an increase in the patient's hemoglobin levels, and an increase in the patient's platelet levels, thereby indicating that the number of multiple myeloma cells within the patient has been reduced.

In general, this document features a method for treating a human having multiple myeloma. The method comprises, or consists essentially of, administering, to the human, an albumin-bound paclitaxel composition under conditions wherein the number of multiple myeloma cells within the human is reduced. Between about 50 mg and 150 mg of the composition can be administered. The composition can be administered at least once a week for at least three weeks. The composition can be administered under conditions wherein the number of multiple myeloma cells within the human is reduced by greater than 10 percent. The composition can be administered under conditions wherein the number of multiple myeloma cells within the human is reduced by greater than 20 percent. Between about 90 mg and 110 mg of the composition per m² can be administered.

In another aspect, this document features a method for treating a human having multiple myeloma. The method comprises, or consists essentially of, administering, to the human, an albumin-bound paclitaxel composition under conditions wherein the level of free lambda light chain polypeptides in the blood of the human is reduced. Between about 50 mg and 150 mg of the composition can be administered. The composition can be administered at least once a week for at least three weeks. The composition can be administered under conditions wherein the level of free lambda light chain polypeptides in the blood of the human is reduced by greater than 10 percent. The composition can be administered under conditions wherein the level of free lambda light chain polypeptides in the blood of the human is reduced by greater than 20 percent. Between about 90 mg and 110 mg of the composition per m² can be administered.

In another aspect, this document features a method for treating a human having multiple myeloma, wherein the method comprises, or consists essentially of, administering, to the human, an albumin-bound paclitaxel composition under conditions wherein the level of IgA in the blood of the human is reduced. Between about 50 mg and 150 mg of the composition can be administered. The composition can be administered at least once a week for at least three weeks. The composition can be administered under conditions wherein the level of IgA in the blood of the human is reduced by greater than 10 percent. The composition can be administered under conditions wherein the level of IgA in the blood of the human is reduced by greater than 20 percent. Between about 90 mg and 110 mg of the composition per m² can be administered.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A is a graph plotting hemoglobin (Hgb) levels (g/dL) for a patient with multiple myeloma and treated with 200 mg (100 mg/m²) of Abraxane® on the days indicated with an arrow.

FIG. 1B is a graph plotting platelet levels (×10⁹/L) for a patient with multiple myeloma and treated with 200 mg (100 mg/m²) of Abraxane® on the days indicated with an arrow.

FIG. 1C is a graph plotting the level free lambda light chain polypeptides (mg/dL) for a patient with multiple myeloma and treated with 200 mg (100 mg/m²) of Abraxane® on the days indicated with an arrow.

FIG. 1D is a graph plotting IgA levels (g/dL) for a patient with multiple myeloma and treated with 200 mg (100 mg/m²) of Abraxane® on the days indicated with an arrow.

### DETAILED DESCRIPTION

This document relates to methods and materials for treating multiple myeloma. For example, this document provides methods and materials for using an albumin-bound paclitaxel composition (e.g., Abraxane®, a paclitaxel albumin-stabilized nanoparticle formulation) to treat multiple myeloma. Paclitaxel is a chemical compound having the following formula: C₄₇H₅₁NO₁₄ (see, e.g., CAS No. 33069-62-4). Paclitaxel can be formulated to be in an albumin-bound form as described elsewhere (see, e.g., Pat. Nos. 5,439,686, 5,498,421, and 6,096,331). In some cases, an albumin-bound paclitaxel composition can include human albumin (e.g., human albumin according to CAS No. 70024-90-7). For example, an albumin-bound paclitaxel composition such as Abraxane® (Abraxis BioSciences Inc.; Los Angeles, Calif.) can be used to treat multiple myeloma. In some cases, an albumin-bound paclitaxel composition (e.g., Abraxane® can be administered to a patient having multiple myeloma to reduce number of multiple myeloma cells within the patient or to reduce the progression of multiple myeloma within the patient. For example, as described herein, administration of Abraxane® to a human patient with multiple myeloma can result in a reduction in the level of free lambda light chain polypeptides in the patient's blood, a reduction in the level of monoclonal IgA polypeptides in the patient's blood, an increase in the patient's hemoglobin levels, and/or an increase in the patient's platelet levels, thereby indicating that the number of multiple myeloma cells within the patient was reduced.

Any appropriate method can be used to identify a patient as having multiple myeloma. For example, standard cancer diagnostic test such as serum protein electrophoresis, quantitative immunoglobulins, serum free light chains, and a direct bone marrow examination can be used to identify a patient as having multiple myeloma.

Once a patient is identified as having multiple myeloma, any appropriate amount of an albumin-bound paclitaxel composition (e.g., Abraxane®) can be administered to the patient intravenously. For example, from about 50 mg/m² to about 300 mg/m² of an albumin-bound paclitaxel composition (e.g., Abraxane®) can be administered intravenously to a patient having multiple myeloma over from about 10 minutes to about six hours (e.g., about 30 minutes) every week to eight weeks (e.g., about once a week). Once an albumin-bound paclitaxel composition is administered to the patient, the patient can be monitored for anti-cancer responses and/or possible adverse effects (e.g., severe neutropenia or severe sensory neuropathy). If adverse effects are observed, then the dose for subsequent administrations can be reduced accordingly. For example, if severe neutropenia is observed, then the subsequent administration can be reduced by, for example, from 25 to 85 percent (e.g., about 50 percent).

Any appropriate method can be used to obtain an albumin-bound paclitaxel composition. For example, an albumin-bound paclitaxel composition can be obtained as described elsewhere (see, e.g., Pat. Nos. 5,439,686, 5,498,421, and 6,096,331). In some cases, an albumin-bound paclitaxel composition such as Abraxane® can be obtained commercially from Abraxis BioSciences Inc. (Los Angeles, Calif.).

The invention will be further described in the following examples, which do not limit the scope of the invention described in the claims.

### EXAMPLES

### Example 1

### Method of Treating Multiple Myeloma

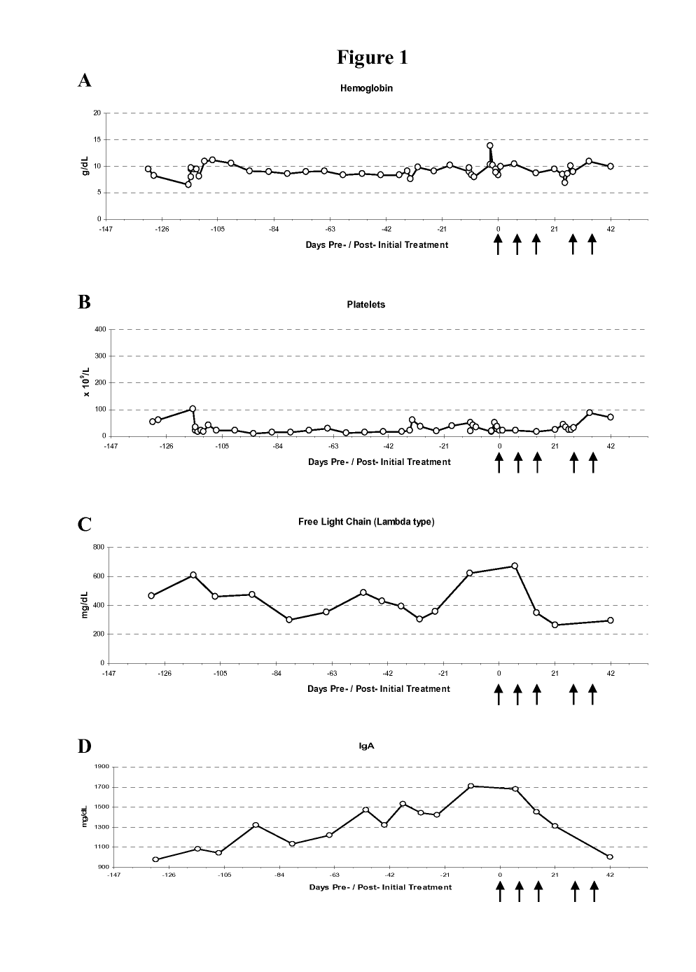

A male patient (aged 68) with multiple myeloma was treated with 200 mg of Abraxane® intravenously over 30 minutes weekly. The patient had renal failure and hypercalcemia with altered mental status prior to starting treatment. He had failed all viable options for further treatment of his myeloma. FIGS. 1A and 1D demonstrate that the patient had a very quick drop in his serum free light chains (lambda type) and IgA in response to intravenously administered Abraxane®. Both of these are polypeptides that can be routinely measured in the serum and serve as markers for disease response to a treatment.

By virtue of them going down, the patient responded to the treatment. Other beneficial effects were noted including a rise in his platelet counts (FIG. 1B) as well as his hemoglobin (FIG. 1C), indicating recovery of the patient's bone marrow.

These results demonstrate that the patient has a disease response. Most patients are usually evaluated for best response after periods of time of at least four months, so it is likely this patient's response could still be improved. Furthermore, it is likely that this patient would have died in the absence of treatment within a few days of his hypercalcemia episode, which occurred shortly before treatment began.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating a human having multiple myeloma, wherein said method comprises administering, to said human, an albumin-bound paclitaxel composition under conditions wherein the number of multiple myeloma cells within said human is reduced.

2. The method of claim 1, wherein between about 50 mg and 150 mg of said composition is administered.

3. The method of claim 1, wherein said composition is administered at least once a week for at least three weeks.

4. The method of claim 1, wherein said composition is administered under conditions wherein the number of multiple myeloma cells within said human is reduced by greater than 10 percent.

5. The method of claim 1, wherein said composition is administered under conditions wherein the number of multiple myeloma cells within said human is reduced by greater than 20 percent.

6. The method of claim 1, wherein between about 90 mg and 110 mg of said composition per m² is administered.

7. A method for treating a human having multiple myeloma, wherein said method comprises administering, to said human, an albumin-bound paclitaxel composition under conditions wherein the level of free lambda light chain polypeptides in the blood of said human is reduced.

8. The method of claim 7, wherein between about 50 mg and 150 mg of said composition is administered.

9. The method of claim 7, wherein said composition is administered at least once a week for at least three weeks.

10. The method of claim 7, wherein said composition is administered under conditions wherein the level of free lambda light chain polypeptides in the blood of said human is reduced by greater than 10 percent.

11. The method of claim 7, wherein said composition is administered under conditions wherein the level of free lambda light chain polypeptides in the blood of said human is reduced by greater than 20 percent.

12. The method of claim 7, wherein between about 90 mg and 110 mg of said composition per m² is administered.

13. A method for treating a human having multiple myeloma, wherein said method comprises administering, to said human, an albumin-bound paclitaxel composition under conditions wherein the level of IgA in the blood of said human is reduced.

14. The method of claim 13, wherein between about 50 mg and 150 mg of said composition is administered.

15. The method of claim 13, wherein said composition is administered at least once a week for at least three weeks.

16. The method of claim 13, wherein said composition is administered under conditions wherein the level of IgA in the blood of said human is reduced by greater than 10 percent.

17. The method of claim 13, wherein said composition is administered under conditions wherein the level of IgA in the blood of said human is reduced by greater than 20 percent.

18. The method of claim 13, wherein between about 90 mg and 110 mg of said composition per m² is administered.
